(19) United States
(12) Patent Application Publication
(10) Pub. No.: US 2023/0348938 A1
(43) Pub. Date: Nov. 2, 2023

(54) METHODS AND APPARATUSES FOR BIOMOLECULE DELIVERY TO PRIMARY HUMAN HEMATOPOIETIC STEM CELLS USING NANOSTRAWS

(71) Applicant: NAVAN Technologies, Inc., Palo Alto, CA (US)

(72) Inventors: Karl Martin HJORT, Lund (SE); Jonas LARSSON, Lund (SE); Ludwig SCHMIDERER, Lund (SE)

(21) Appl. No.: 18/041,609

(22) PCT Filed: Aug. 16, 2021
(86) PCT No.: PCT/US2021/046090

§ 371 Date: Feb. 14, 2023

Related U.S. Application Data
(60) Provisional application No. 63/065,922, filed on Aug. 14, 2020.
(60) Provisional application No. 63/152,601, filed on Feb. 23, 2021.

(51) Int. Cl. C12N 15/89; C12N 13/00; C12N 15/113; A61K 35/28

(52) U.S. Cl. CPC C12N 15/89; C12N 13/00; C12N 15/113; A61K 35/28; C12N 2310/14

(57) ABSTRACT

Described herein are methods and devices useful for delivering a biologically relevant cargo to biological cells, especially to sensitive cells such as primary stem cells and primary derived cells. The methods and devices may use centrifugation of cells to drive the cells into hollow nano straws on a substrate for delivering the biologically relevant cargo to the cells.

### CROSS REFERENCE TO RELATED APPLICATIONS

This patent application claims priority to U.S. provisional patent application no. 63/065,922, titled “METHODS AND APPARATUSES FOR BIOMOLECULE DELIVERY TO PRIMARY HUMAN HEMATOPOIETIC STEM CELLS USING NANOSTRAWS, filed on Aug. 14, 2020, and U.S. provisional patent application No. 63/152,601, titled “METHODS AND APPARATUSES FOR BIOMOLECULE DELIVERY TO PRIMARY HUMAN HEMATOPOIETIC STEM CELLS USING NANOSTRAWS,” filed on Feb. 23, 2021, each of which are herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Devices and methods for delivering biologically relevant cargo to cells.

### BACKGROUND

Hematopoietic stem cells (HSCs) give rise to all different blood cells throughout life. They are used therapeutically in bone marrow transplantations for hematological malignancies and congenital diseases that affect the blood system. Just as other primary cell types, human hematopoietic stem and progenitor cells (HSPCs) are a scarce resource, and the number of cells that can be obtained is a limiting factor for clinical applications, such as transplantations, and functional assays. HSCs are targeted with nucleic acids for gene therapy purposes, and for functional studies to elucidate regulatory mechanisms of HSCs, such as self-renewal, differentiation, and malignant transformation. In such studies, foreign genetic material is conventionally introduced by viral transduction or electroporation. Integrating viruses, such as retro- and lentivirus have low transduction efficiencies, especially when aiming for a single integration event per cell. They also carry the risk of insertional mutagenesis, which can turn the treated cells into malignant, leukemia causing cells. Electroporation is very efficient, but it has been reported to cause severe damage and functional impairment in HSPCs. Since low cell numbers are already a limiting factor for clinical use and in research applications, a non-perturbative gene delivery method, with less impact on the cell viability and function, is acutely needed. Described herein are methods that may address these and other issues.

### SUMMARY OF THE DISCLOSURE

Described herein are methods, apparatuses and compositions (e.g., kits) for introducing exogenous material, including genetic material, into stem cells using nanostraws. In particular, described herein are methods and apparatuses for transfecting cells, such as hematopoietic stem cells to enhance engraftment.

Introduction of exogenous genetic material into primary stem cells is essential for studying biological function and for clinical applications. Traditional delivery methods for nucleic acids, such as electroporation, have advanced the field, but have negative effects on stem cell function and viability. Primary stem cells are difficult to transfect, and their viability and function are impaired by traditional transfection methods. As described herein, nanostraws can be used to deliver RNA to primary human hematopoietic stem cells without any detectable negative effects. In some examples, nanostraw treated cells show no alterations in gene expression, and fully retain their proliferative capacity and their potential to regenerate blood cells of different lineages in vivo. This non-perturbative transfection method can benefit functional studies and clinical applications where minimal impact on stem cell function and viability is required. For example, the methods and apparatuses described herein may be used to enhance engraftment of stem cells, e.g., hematopoietic stem cells. The methods and apparatuses described herein may also be used for primary derived cells (e.g., induced pluripotent stem cells, iPSCs, which may be derived from a primary cell).

As described herein, nanostraws may be used to efficiently deliver molecular cargos to cells with minimal effects on cell viability. Nanostraws include hollow aluminum oxide tubes (common diameter of 100-200 nm, length 1-3 μm) that are embedded in a cell culture compatible polymer membrane. The nanostraws form a direct fluidic pathway from a cargo containing compartment beneath the nanostraw-membrane into the cytoplasm of cells cultured on top of the membrane. Adherent cells may be cultured on top of nanostraws and may pull themselves down onto the nanostraws, resulting in the nanostraws piercing through the cell membrane. The cargo is delivered into the cells by passive diffusion or electrokinetically driven by a weak, pulsed electric field. Nanostraws target small molecules, RNA, DNA, and proteins to different adherent cells in a tunable, non-toxic, and highly efficient manner. Also described herein are methods and apparatuses for using nanostraws to gain intracellular access to primary cells in suspension, which has not previously been possible.

Nanostraws assisted transfection as described herein may be used for RNA delivery to human hematopoietic stem and progenitor cells (HSPCs). Here, we establish a nanostraw based transfection system for non-adherent primary cell types, Centrifugation enhanced Nanostraw Transfection (CeNT). CeNT can be used as an efficient tool for small molecule and RNA delivery to human cord blood-derived CD34+ HSPCs. CeNT is exceptionally gentle on this sensitive primary cell type, and can have no detectable effects on HSPC functionality and gene expression, making it an attractive alternative to other, more disruptive transfection methods. Although CeNT is one method of using nanostraws to transfect cells (e.g., stem cells, including but not limited to hematopoietic stem cells), other nanostraw transfection methods may be used, including culturing cells onto a substrate including the nanostraws.

Nanostraws may include hollow alumina nanotubes that can be used to deliver biomolecules to living cells. Nanostraws may be used to target human primary HSPCs and show efficient delivery of mRNA, short interfering RNAs (siRNAs), DNA oligonucleotides, and dextrans of sizes ranging from 6 kDa to 2000 kDa. As described herein, nanostraw treated cells were fully functional and viable, with no impairment in their proliferative or colony forming capacity, and showed similar long-term engraftment potential in vivo as untreated cells. Additionally, gene expression of the cells was not perturbed by nanostraw treatment, while conventional electroporation changed the expression of more than 2000 genes. The results shown herein demonstrate that nanostraw mediated transfection is a gentle alternative to established gene delivery methods, and uniquely suited for non-perturbative treatment of sensitive primary stem cells.

The methods and apparatuses described herein provide optimized conditions for efficiently delivery biomolecules to stem cells (including, but not limited to hematopoietic stem cells). In particular, described herein are methods and apparatuses that specify nanostraw dimensions (e.g., lengths, diameters, etc.) that perform significantly, and unexpectedly, better than other dimensions, and/or specific voltages that outperform other voltages. In general, the methods and apparatuses described herein included shorter than usual nanostraws (e.g., 1.2 μm or smaller, 1.1 μm or smaller, between 200 nm-1.2 μm, between 500 nm-1.2 μm, etc.) extending from (and embedded in) a membrane having a mechanical strength at fixed film thickness of greater than that of polycarbonate (e.g., using PET), and critically, centrifuging the stem cells onto the nanostraws, and applying a voltage of greater than 35 V (e.g., greater than 37 V, greater than 38 V, greater than 39 V, 40 V or greater, between 35 and 60 V, between 35 and 55 V, between 35 and 50 V, between 35 and 45 V, between 37 and 42 V, etc.) to electrokinetically drive cargo (typically a cargo of between 5 and 2500 kDa, between 5 and 2100 kDa, between 5 and 2000 kDa, between 6 and 2000 kDa, etc.) into at least 20% of the stem cells. Compare, for example, to U.S. patent application Ser. No. 16/038,062, titled “APPARATUSES AND METHODS USING NANOSTRAWS TO DELIVER BIOLOGICALLY RELEVANT CARGO INTO NON-ADHERENT CELLS” (filed on Jul. 17, 2018), herein incorporated by reference in its entirety. Surprisingly, the higher voltage (e.g., 30V or greater, 35 V or greater, etc.), resulted in higher cell viability and higher transfection efficacy as compared to lower voltages (e.g., less than 30 V). This improvement appears to be a function of both the specific length range (e.g., between 200 nm and 1.1 μm) as well as the voltage applied. Outside of this range, the transfection efficacy and survivability was significantly lower. For example, when using stem cells (e.g., hematopoietic stem cells) if longer nanostraws are used and/or if lower voltages are used, the viability and/or number of cells that take up cargo (e.g., cargo between 5 and 2000 kDa, such as siRNA), falls off dramatically.

For example, described herein are methods (and systems configured to perform these methods) of delivering a biologically relevant cargo, the method comprising: centrifuging primary stem cells to drive the primary stem cells into contact with a plurality of nanostraws, wherein the plurality of nanostraws extend through a substrate and a distance beyond the substrate that is between 200 nm and 1100 nm, further wherein the plurality of nanostraws are hollow and have an inner diameter of between 5 nm-500 nm; and driving the cargo from the nanostraws into an intracellular volume of the cells with a pulsed electrical field comprising at least 30 volts across the cells, so that at least 80% of the cells are viable and at least 20% of the cells take up the biologically relevant cargo.

Any appropriate primary stem cells may be used, e.g., the primary stem cells may be isolated hematopoietic stem cells. In some variations the membrane may comprise polyethylene terephthalate (PET) (or any other membrane having a strength that is greater than polycarbonate). As mentioned above, these methods and apparatuses may also be used with primary derived cells.

In some variations, the membrane may have an areal density of at least 1*10⁷/cm² (e.g., of between 2*10⁷/cm² and 4*10⁷/cm²).

The nanostraws may have a diameter that is less than 500 nm (e.g., less than 200 nm, between 50 nm-200 nm, etc.).

The methods described herein may be particularly well suited for cargo having a weight of between about 5 and 2000 kDa.

Any of the methods and apparatuses described herein may also or alternatively include a step of changing the media within x minutes after delivery (via nanostraw as described herein) of the cargo, e.g., within x minutes of applying the pulsed electrical field. In some variations x may be 300 minutes, 240 minutes, 180 minutes, 120 minutes, 60 minutes, 45 minutes, 40 minutes, 30 minutes, 25 minutes, 20 minutes, 15 minutes, 10 minutes, etc. For example, the media may be changed within 20 minutes or less. In some variations, the media may be changed within 3 days after delivery, within 2 days, within 1 day, etc.

In any of the methods and apparatuses (e.g., systems) described herein the cells, such as, but not limited to CD34+ hematopoietic stem cells, may be transfected on the same day that they have been purified/obtained with a high degree of success (e.g., 50% or greater, 55% or greater, 60% or greater, 65% or greater, 70% or greater, 75% or greater, 80% or greater, 85% or greater, 90% or greater, 95% or greater, etc. survival and/or expression). This is particularly remarkable given the lack of success using other known delivery/transfection methods. Thus, in general, hematopoietic stem cell may be transfected using nanostraws as described herein immediately or nearly immediately (e.g., within about 24 hours, within about 22 hours, within about 20 hours, within about 18 hours, within about 16 hours, within about 14 hours, within about 12 hours, within about 10 hours, within about 9 hours, within about 8 hours, within about 7 hours, within about 6 hours, within about 5 hours, within about 4 hours, within about 3 hours, within about 2 hours, within about 1 hour, etc.) of isolation of the cells, e.g., hematopoietic stem cells. Without being bound by theory, the gentle nature of the nanostraw delivery techniques described herein may allow transfection of cells (e.g., HSPCs) on the same day as they have been purified/obtained. This is in stark contrast to electroporation which has been shown to give poor transfection results on the same day as the cells have been purified, presumably due to the cell stress induced by the purification process combined with the stress from the electroporation process. See, e.g., Wu et al. Gene Therapy volume 8, pages 384-390(2001). Thus, surprisingly, the methods described herein may allow for high efficiency transfection of cells, and in particular, HSPCs, on the same day as the cells are obtained/purified.

As mentioned, the methods and apparatuses (e.g., systems including nanostraws) may be used to enhance engraftment. For example, the cells may include hematopoietic stem cells. The cargo may be any one or more compounds/compositions that may enhance engraftment of hematopoietic stem cells, such as cell penetrating peptides, proteins, mRNA, siRNA, miRNA, sugars, DNA, small molecules, and cardiac specific transcription factors. The cells may also be treated (using nanostraws) to include one or more normal or modified copies of a deficient gene that may be introduced into patient's own HSCs to reestablish effective gene function.

There are a number of ways to enhance HSC engraftment. The cells described herein may be modified by the nanostraws to improve or enhance the proliferative capacity of the cells and/or responsiveness of the cells (e.g., HSPCs) to homing gradients and/or to increase or improve the tethering of transplanted cells (e.g., HSPCs) to the target region, such as basement membrane (BM) endothelium and subsequently their adhesion, e.g., in the BM microenvironment.

For example, any of these methods may include collecting or receiving cells (e.g., hematopoietic stem/progenitor cells) from the patient or donor(s), and treating the cells with nanostraws as described herein in order to enhance engraftment and/or to express one or more therapeutic molecule. These cells may then be screened and some or all of them may be delivered to a patient as part of a therapy.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A-a shows a 30° tilted view scanning electron microscope (SEM) picture of a nanostraw membrane. FIG. 1A-b shows a close up version of one of the nanostraws shown in FIG. 1A-1.

FIG. 1B shows a schematic overview of the Centrifugal enhanced Nanostraw Transfection (CeNT) procedure described herein including applying an external force to cells by centrifugation to press cells onto a nanostraw membrane with nanostraws. Cargo is delivered into CD34+ HSPCs through nanostraws by application of a gentle, pulsed electric field.

FIG. 1C shows a 45° tilted view SEM picture of CD34+ cell on nanostraw membrane.

FIG. 1D-a-FIG. 1D-d shows fluorescent microscopy images of propidium iodide (PI) delivery to CD34+ cells with CeNT. On the left, PI in the medium with the cells. On the right, the PI is in the cargo compartment. FIG. 1D-a (control) and FIG. 1D-b (nanostraw) show cell images. FIG. 1D-c (control) and FIG. 1D-d (nanostraw) show PI imaging.

FIG. 1E shows representative FACS plot of CD34+ cells that were exposed to Green Fluorescent Protein (GFP) mRNA through CeNT, 6 h post treatment. The left panel in FIG. 1E shows an untreated control and the right panel in FIG. 1E shows treated cells expressing GFP.

FIG. 1F shows qPCR analysis of RNA knock-down after delivery of different STAG2 siRNAs to CD34+ hematopoietic stem/progenitor cells using CeRT. Relative STAG2 expression levels 2 days after siRNA treatment with CeNT. siRNA1 and siRNA2 target STAG2, and siRNA3 is a control with no specific target (n=3).

FIG. 1G shows results from flow cytometry analysis of delivery of different sized oligonucleotides into CD34+ HSPCs. FITC labeled oligonucleotides were delivered to the cells using the nanostraws and delivery methods described herein

FIG. 1H-a-FIG. 1H-d show efficiency of CeNT mediated DNA oligo delivery to HSPCs (n=3). HSPCs that were targeted with FITC-labelled Dextran (6 kDa) using CeNT FIGS. 1H-a-FIG. 1H-d shows imaging results from cells. The top panels show bright field images of all cells in a field and the bottom panels show fluorescent microscopy images indicative of the fluorescent dextran cargo. The left panels (FIG. 1H-a and FIG. 1H-c) are untreated. The right panels (FIG. 1H-b and FIG. 1H-d) show cells treated with 6 kDa fluorescent dextran.

FIG. 1I shows delivery efficiency of FITC-labelled dextrans of different sizes (6 kDa to 2000 kDa) to HSPCs using CeNT (n=3) compared with an untreated control.

FIG. 2A schematically illustrates the experimental protocol for determining the effect of using the nanostraws and CeNT procedure described herein for delivering cargo to CD34+ cells and comparing it with electroporation. After treatment, cells are cultures, sorted, and assayed. Assay results are shown in FIGS. 2B-2H.

FIG. 2B shows results from continued culture assays as described in FIG. 2A using CeNT and comparing it with results from electroporation. FIG. 2B shows fold expansion of cells treated with different conditions after 2 weeks in culture (n=9, 3 biological and 3 technical replicates). Below the x-axis, GFP— denotes mock treatment without mRNA.

FIG. 2C shows results from colony forming cell (CFC) assays as described in FIG. 2A using CeNT and comparing it with results from electroporation for assaying the ability of CD34+ HSPCs to proliferate and differentiate. FIG. 2C shows the number of colonies derived from sorted, live CD34+GFP+ or CD34+GFP− cells after 2 weeks in culture (n=8-9, 3 biological and 2-3 technical replicates).

FIG. 2D shows results from microarray analyses for assaying affect of CeNT on gene expression on CD34+ HSPCs. FIG. 2D shows that GFP CeNT treated cells, unlike electroporated cells, are clustered together closely with untreated cells in the analysis. Principal component analysis of sorted, live CD34+GFP+/− cells (n=3).

FIG. 2E shows volcano plots of CeNT and electroporation conditions compared to untreated cells 6 h after treatment (n=3) to show statistical significance (P value) vs magnitude of change (fold change). GFP is circled.

FIG. 2F shows graphical results of the number of differentially expressed genes from a differential gene analysis on CD34+ HSPCs performed as described in FIG. 2A. Mock and GFP nanostraw and electroporation treatments were performed. No significantly upregulated or downregulated genes were detected in response to mock nanostraw treatment. Number of differentially expressed genes (fold Change>1.5, adj. p<0.05).

FIG. 2G shows a Venn diagram graphical comparison of the differential gene expression results shown in FIG. 2F illustrating overlap and non-overlap of gene expression after nanostraw treatment with GFP, and mock electroporation treatment, and electroporation treatment with GFP were performed.

FIG. 2H shows a pathway enrichment analysis diagram of upregulated genes from the results shown in FIG. 2F for different response mechanisms such as interferon signaling with nanostraw and electroporation treatments with GFP and mock treated without GFP.

FIG. 3A schematically illustrates how CeNT treated hematopoietic stem cells described herein can be used to successfully engraft in mice. FIG. 3A shows the nanostraws and methods described are used with CD34+ hematopoietic stem/progenitor cells and the cells injected in vivo into sub-lethally irradiated, immunocompromised mice. Blood, bone marrow, or other samples from the mice have been analyzed. In the CeNT GFP condition, only GFP+ cells were sorted and transplanted. Results are shown in FIG. 3B-FIG. 3E.

FIG. 3B shows graphical results of peripheral blood analysis 4 months post-transplantation from mice injected with human CD34+ hematopoietic stem/progenitor cells mice using CeNT as shown in FIG. 3A or left untreated (n=3-4). The origin of the cells in the peripheral blood sample as human or mouse was determined and the ratio calculated as the percent chimerism in peripheral blood.

FIG. 3C shows graphical results of bone marrow analysis from mice injected with human CD34+ hematopoietic stem/progenitor cells mice treated using Cert or untreated, similar to as described in FIG. 3B. (n=3-4). The origin of the cells in the bone marrow sample as human or mouse was determined and the ratio calculated and plotted as the percent chimerism in bone marrow.

FIG. 3D shows a representative FACS plot of bone marrow cells showing the frequency of human CD45+ cells in mouse bone marrow 4 months post-transplantation.

FIG. 3E shows graphical results of bone marrow analysis from mice injected with human CD34+ hematopoietic stem/progenitor cells mice treated using CeRT, similar to as described in FIG. 3C. The lineage of the cells in the bone marrow samples as lymphoid cells (CD19+ B-cells and CD3+ T-cells) and myeloid (CD33+) cells within the human CD45+ population in the mouse bone marrow and the ratio calculated and plotted.

FIG. 4A-FIG. 4C show the effect of nanostraw length and cargo size on delivery efficiency. FIG. 4A shows 30° tilted view SEM images of nanostraws of different lengths. Scalebars denote 200 nm. FIG. 4B shows percentage of GFP+ HSPCs 1 day after mRNA delivery using differently sized nanostraw. FIG. 4C shows viability (7AAD− & Annexin V−) of the HSPCs shown in FIG. 4B. n=2-3 in FIG. 4B and FIG. 4C.

FIG. 5A-FIG. 5B shows results from cargo delivery to HSPCs using the methods described herein. FIG. 5A shows percentage of live (7AAD−) HSPCs immediately after they were subjected to CeNT mediated delivery of DNA oligonucleotides. FIG. 5B shows percentage of live (7AAD−) HSPCs immediately after they were subjected to CeNT mediated delivery of dextrans of different sizes. (n=3).

FIG. 6A-FIG. 6C shows results from cargo delivery to HSPCs using the methods described herein compared with cargo delivery using conventional electroporation. FIG. 6Aa-FIG. 6Ae show representative FACS plots of live CD34+ cells treated with different mRNA or mock conditions. FIG. 6B shows percentage of viable (7AAD− and Annexin V−) HSPCs at 1 day and FIG. 6C shows percentages 2 days after CeNT treatment or conventional electroporation (n=3).

FIG. 7A-FIG. 7C shows representative FACS plots of populations (CD34+GFP+ or CD34+GFP−) that were sorted for the transplantation experiment shown in FIG. 3A. The gates of the sorted populations are at the bottom (FIG. 7A), top (FIG. 7B) and bottom (FIG. 7C).

FIGS. 8A-8B are graphs illustrating the effect of changing the media shortly vs. not changing the cell media immediately after delivery of a cargo (e.g., GFP) using nanostraws as described herein. In FIG. 8A the graph shows a comparison of cell viability (total live cells) following identical delivery of cargo (e.g., GPF) using nanostraws either without changing the media or with a change in media within 15 minutes of performing the procedure. FIG. 8B shows that the expression of GFP in both situations (normalized by cell) is essentially identical.

FIG. 9 is a graph illustrating the effect of buffer pH on the methods described herein.

### DETAILED DESCRIPTION

Described herein are nanostraw apparatuses (devices) and methods of using them in order to deliver biologically relevant cargo to primary stem cells, such as hematopoietic stem cells. Primary stem cells containing biologically relevant cargo may be especially useful in laboratory or clinical use and both the natural functions of a primary stem cell and the functions of the biologically relevant cargo are relevant. Primary stem cells from a biological tissue sample can be rare, difficult or expensive to obtain, and sensitive to disruption. The unique functions of primary stem cells are not readily replaced by a different type of cell. Maintaining the normal functions of the primary stem cells, such as normal gene expression and metabolic functioning without unwanted non-specific effects, along with specific activity of the biologically relevant cargo, can require a delicate balancing. Described herein are nanostraw apparatuses (devices) and methods of using them in order to maintain normal functions of the primary stem cells, such as normal gene expression without unwanted non-specific effects, while delivering specific activity of the biologically relevant cargo. Although many of the examples described herein are specific to primary stem cells (e.g., primary hematopoietic stem cells), the methods and apparatuses described herein may also or additionally be used for primary derived cells (also referred to as derived primary cells), such as iPSCs.

Described herein are methods of delivering a biologically relevant cargo including the steps of obtaining primary stem cells (e.g., hematopoietic stem cells), centrifuging the hematopoietic stem cells to drive the hematopoietic stem cells into contact with the plurality of nanostraws, and driving the cargo from the nanostraws into an intracellular volume of the cells with a pulsed electrical field. Centrifugation can be used to make the cells come in contact with nanostraws and allow for intracellular delivery of biologically relevant molecules into the intracellular space. For this method, centrifugation may be performed when the cells are in a container incorporating a nanostraw membrane. Once the container with the cells is centrifuged, the cells may be pelleted onto the nanostraws, allowing for intracellular delivery. Combining this centrifugation step with other steps and a selected nanostraw platform can deliver a biologically relevant cargo to primary stem cells while maintaining primary stem cell functions. This combination may be referred to herein as Centrifugation enhanced Nanostraw Transfection (CeNT) and it is an attractive alternative to other, more disruptive transfection methods.

### Definitions

Biologically relevant cargo includes small and large particles such as nucleic acid (DNA, RNA), lipids, and protein.

Hematological malignancy refers to cancers that affect the blood, bone marrow, and lymph nodes, such as various types of leukemia (acute lymphocytic (ALL), chronic lymphocytic (CLL), acute myeloid (AML), chronic myeloid (CML)), myeloma, and lymphoma (Hodgkin's and non-Hodgkin's (NHL)).

Hematopoietic stem and progenitor cell (HSPC) refers to diverse population of cells responsible for maintaining and regenerating the hematopoietic system. HSPCs can be isolated from bone marrow (BM), peripheral blood (PB), placenta, spleen, and umbilical cord blood.

Isolating a cell includes a process of dissociating or otherwise removing a cell from a tissue sample (e.g., blood tissue, placental tissue), and separating the cell from other cells or non-cells in the tissue. Isolated cells will generally be free from contamination by other cell types and will generally be able to be propagated and expanded.

An isolated cell e.g., “isolated stem cell,” includes a cell that is substantially separated from other, different cells of the tissue, e.g., blood or placenta from which the stem cell is derived. A stem cell is “isolated” if at least 50%, 60%, 70%, 80%, 90%, 95%, or at least 99% of the cells with which the population of cells, or cells from which the population of cells is derived, is naturally associated, i.e., stem cells displaying a different marker profile, are removed from the stem cell, e.g., during collection and/or culture of the stem cell. In some embodiments, an isolated cell exists in the presence of a small fraction of other cell types that do not interfere with the utilization of the cell for analysis, production or expansion of the cells. A population of isolated cells can be at least 30%, 40%, 50%, 60%, 70%, 80%, 85%, 90%, 95%, 98%, or 99% pure, or any interval thereof. In a specific embodiment, a population of isolated cells are at least 98% or at least 99% pure. As used herein, the term “population of isolated cells” means a population of cells that is substantially separated from other cells of a tissue, e.g., placenta, from which the population of cells is derived.

A multipotent cell refers to a cell that has the ability to differentiate into some, though not necessarily all, types of cells of the body, or into cells having characteristics of some, but not all, types of cells of the body. In certain embodiments, for example, an isolated placental cell that has the capacity to differentiate into a cell having characteristics of neurogenic, chondrogenic and/or osteogenic cells is a multipotent cell.

Nanostraw refers to hollow inorganic nanotubes that may be formed from nanoporous polymer films. These nanostraws can pierce a cell membrane, enabling direct delivery of biological or chemical cargoes through the nanostraws and into the cell. This process largely avoids biological challenges related to endocytosis and degradation, and is largely agnostic to the cargo type (e.g., DNA, RNA, proteins, and small molecules, etc.).

A nanostraw membrane (a nanoporous substrate) can have a plurality of nanostraws. A nanostraw membrane may be fabricated from a track-etched membrane. In some variations, a nanostraw membrane as described herein may have a higher mechanical strength at fixed film thickness than polycarbonate. A nanostraw membrane of interest is polyethylene terephthalate (PET or PETE).

Primary cells refers to cells obtained from tissue, such as from an animal or subject.

siRNA: Small interfering RNAs (siRNA) are double stranded RNA molecules, generally 20-25 base pairs long which play a role in RNA interference (RNAi) by interfering with the expression of specific genes with complementary nucleotide sequence. Interfering RNA includes “small-interfering RNA” or “siRNA,” e.g., a small inhibitory RNA duplex that induces gene silencing by operating within the RNA interference (“RNAi”) pathway. These siRNA are dsRNA that can vary in length, and can contain varying degrees of complementarity between the antisense and sense strands, and between the antisense strand and the target sequence. Small interfering RNA can be 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). Some, but not all, siRNA have unpaired overhanging nucleotides on the 5′ and/or 3′ end of the sense strand and/or the antisense strand. Additionally, the term “siRNA” includes duplexes of two separate strands, as well as single strands that can form hairpin structures comprising a duplex region, which may be referred to as short hairpin RNA (“shRNA”). siRNA can be chemically synthesized. siRNA or generated by cleavage of longer dsRNA (e.g., dsRNA greater than about 25 nucleotides in length) such as with E. coli RNase III or Dicer. These enzymes process the dsRNA into biologically active siRNA.

Stem cells refers to cells capable of dividing and renewing themselves, are unspecialized, and can give rise to specialized cell types. Stem cells include epithelial stem cells, hematopoietic stem cells (blood stem cells), mesenchymal stem cells, neural stem cells, and skin stem cells. A stem cell of interest in a hematopoietic stem cell (HSC). The term “pluripotent stem cells” refers to stem cells that has complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “embryonic stem cells” refers to stem cells derived from the inner cell mass of an early stage embryo, e.g., human, that can proliferate in vitro in an undifferentiated state and are pluripotent. The term “bone marrow stem cells” refers to stem cells obtained from or derived from bone marrow. The term “amniotic stem cells” as used herein refers to stem cells collected from amniotic fluid or amniotic membrane. The term “embryonic germ cells” as used herein refers to cells derived from primordial germ cells, which exhibit an embryonic pluripotent cell phenotype.

Described herein are methods of delivering a biologically relevant cargo, the method including obtaining primary stem cells; centrifuging the hematopoietic stem cells to drive the hematopoietic stem cells into contact with the plurality of nanostraws, and driving the cargo from the nanostraws into an intracellular volume of the cells with a pulsed electrical field.

The plurality of nanostraws may extend through a substrate and a distance beyond the substrate that is between 2 nm and 1500 nm, such as between 200 nm and 1100 nm, or between 200 nm and 800 nm, or around 300 nm. The plurality of nanostraws are hollow and can have an inner diameter of between such as 5 nm-500 nm, such as between 50 nm-200 nm, or between 100 nm-130 nm. A substrate (for nanostraws) may be a relatively strong polymer configured to withstand centrifugation pressures and cell pressure and maintain nanostraw shape and integrity, such as an artificial polymer such as polycarbonate or polyethylene terephthalate. A polymer of interest for a substrate is polyethylene terephthalate (PET OR PETE), or polyester. A substrate and nanostraws extending through a substrate may be configured to maintain the nanostraws in a substantially upright configuration (e.g., perpendicular to the substrate). Such a configuration may aid in creating relatively small or right sized openings in the cell membranes and cytoplasm for delivering a biologically relevant cargo without causing undue membrane or cell stress. Such a configuration may aid in minimizing non-specific gene expression. A substrate may be fabricated from a material, such as polyethylene terephthalate material (e.g., PET), with a tensile strength at least 160 MPa, at least 165 Mpa, or about 170 MPa. A nanostraw membrane may be made from a material (e.g., PET) with a flexural strength at least 75 psi, at least 80 psi, etc. An areal density of nanostraws on a substrate may be least 1*10⁷/cm², at least 1.5*10⁷/cm², at least 2*10⁷/cm², at least 3*10⁷/cm²′ at least 4*10⁷/cm², or less than these amounts, such as less than 1*10⁷/cm², less than 1.5*10⁷/cm², less than 2*10⁷/cm², less than 3*10⁷/cm²′ less than 4*10⁷/cm², or between these amounts, such as between 2*10⁷/cm² and 4*10⁷/cm². A nanostraw platform may be fabricated to provide 1-50 nanostraws per cell, 5-40 nanostraws per cell, or 10-20 nanostraws per cell.

Some methods include the step driving the cargo from the nanostraws into an intracellular volume of the cells with a pulsed electrical field comprising at least 20 volts, at least volts, or at least 40 volts across the cells.

In some methods disclosed herein least 20%, at least 30%, at least 40%, at least 50%, at least 60%, at least 70%, at least 80%, at least 90% of the cells (stem cells) take up biologically relevant cargo. In some methods, a biologically relevant cargo includes nucleic acid or protein. Nucleic acids of interest include RNA such as mRNA and siRNA. In some methods, a biologically relevant cargo and stem cell are configured to treat or prevent a genetic disease, such as a hematological malignancy. Some methods include delivering the cells (e.g., stem cells with biologically relevant cargo) to a subject (e.g., a plant, animal or human patient) where stem cell activity and biologically relevant cargo activity is desired. An animal may be a mammal or a non-mammal. Cells (e.g., stem cells with biologically relevant cargo) may be delivered to a body organ or tissue. A tissue of interest is bone marrow. Cells (e.g., stem cells with biologically relevant cargo) may be delivered to a subject by injection, infusion, or transplant. Animals include lab animals (e.g., guinea pig, mouse, rat), farm or other work animals (e.g., cows, horse, goats), zoo animals, and pets (e.g., cat, dogs). A method as described herein may include propagating and/or differentiating cells (e.g., stem cells with biologically relevant cargo) in vitro or in vivo. Stem cells with biologically relevant cargo can be propagated and differentiated into one, two, or more cell types. For example, hematopoietic stem cells can be differentiated into myeloid cells and lymphoid cells.

Some methods are configured to minimize side effects resulting from delivering a biologically relevant cargo. As indicated above, the methods and devices described herein for delivering biologically relevant cargo to sensitive cells (e.g., CeRT with centrifugation, a mechanically strong substrate such as polyethylene terephthalate (PET) substrate, relatively short nanostraws, etc.) can be configured to minimize non-specific effects, such as non-specific gene activation. Non-specific gene or other nucleic acid activation can be assayed by comparing treated cells with untreated cells and assaying for gene expression in the cells such as by using microarrays or qPCR or for protein such as using protein arrays or other proteome analysis. Either populations of cells or individual cells can be assayed. In some examples, assaying for non-specific effects may include assaying expression for a group of genes (e.g., 1-1000, 1-100, 1-50, 1-10, 1-5, 1-3 genes) such as interferon alpha/beta signaling, interferon signaling, interferon gamma signaling, induction of interferon alpha/beta, cellular senescence, chromatin modifying enzymes, chromatin organization, deubiquination, megakaryocyte differentiation and platelet function, negative regulators of DDX58/IFIH1 signaling, pre-NOTCH expression and processing, estrogen dependent gene expression, antiviral mechanism by IFN stimulated genes, and ISG15 antiviral mechanism. Expression of genes in treated cells vs untreated cells can be undetectable, fewer than 1% different, fewer than 2% different, fewer than 3% different, fewer than 4% different, fewer than 5% different.

### Experimental

### Materials and Methods

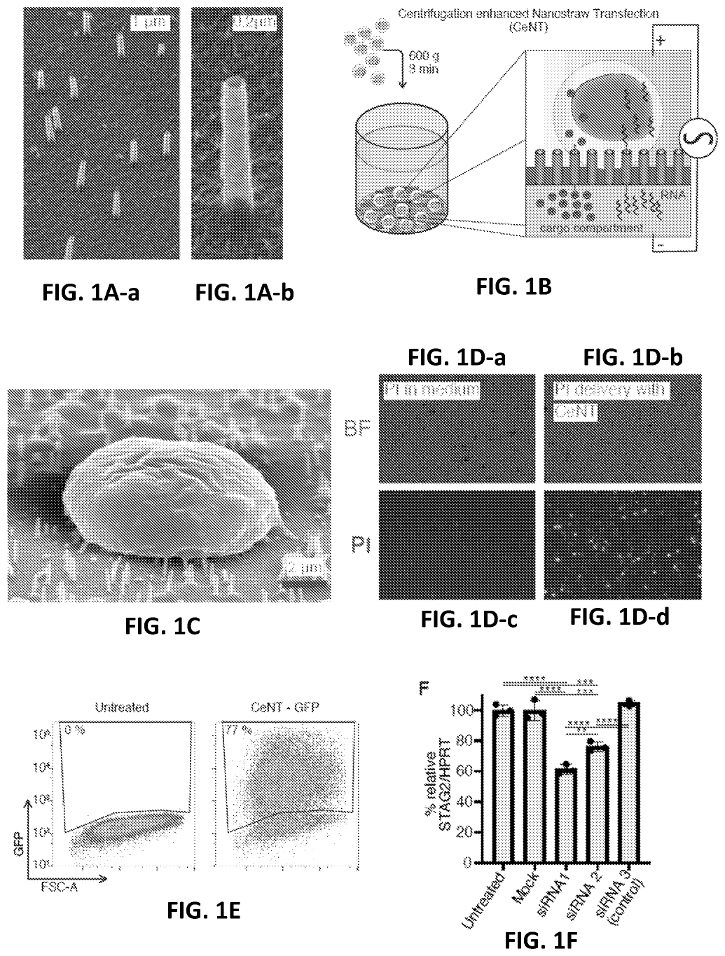

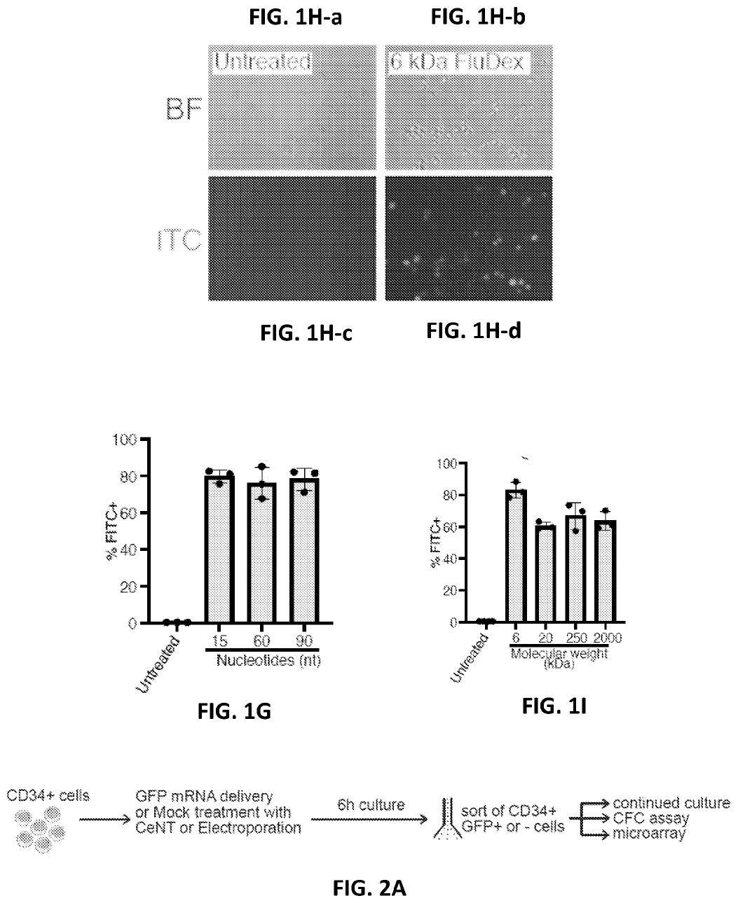

Nanostraws provide intracellular access to primary human hematopoietic stem and progenitor cells. Human primary stem cells, including HSPCs, are challenging to transfect and sensitive to stress that can be caused by the chosen transfection method. We attempted to overcome the challenge of transfecting HSPCs by using nanostraws, which have previously been shown to have low impact on cell function and viability. In order to make nanostraws applicable to non-adherent HSPCs, we produced nanostraws as previously described, with modifications to the geometry and starting-material. The nanostraws were fabricated to have 100-130 nm diameter, approximately 1 μm length, and an areal density of 3*10⁷ cm⁻², which corresponds to about 15 nanostraws per cell with 8 μm diameter (FIG. 1A). The nanostraw membrane was fixed to the bottom of a 5 mm plastic tube with the nanostraws pointing into the tube, forming a nanostraw cell culture container. To mimic the tight attachment to the surface that is necessary for successful transfection, we applied an external force by centrifugation to press the cells directly onto the nanostraw membrane (FIG. 1B). To prevent damage to the nanostraws during centrifugation, we adjusted the geometry of the nanostraw membrane by making shorter (1 μm) nanostraws in a mechanically more stable polymer. To that end, we exchanged the track-etched membrane from polycarbonate (PC) to polyethylene terephthalate (PET), which was found to have a higher mechanical strength at fixed film thickness. We applied a low-power, pulsed square electric field across the cells to locally destabilize the cell membrane just above the nanostraw tips and to electrokinetically drive cargo into the cells. To account for the less close interface between HSPCs and the nanostraws, compared to adherent cells, we applied 40 V instead of the 20 V pulses which are commonly used for adherent cells. Thereby, we developed a relatively quick transfection method, taking approximately 10 min, including the centrifugation step, and termed it Centrifugation enhanced Nanostraw Transfection (CeNT).

To qualitatively assess whether CeNT can provide intracellular access to the cytosol of human CD34+ HSPCs (FIG. 1C), we delivered propidium iodide (PI), which is a small molecule that does not usually cross the membrane of live cells, and only stains dead cells and debris. Upon delivering PI to HSPCs using CeNT, approximately 70% of the cells took up the dye (FIG. 1D). As a negative control, the voltage pulses were applied with PI added directly to the cell culture medium instead of the cargo compartment, which resulted in practically no uptake of the dye (FIG. 1D). The cytosolic injection of PI shows that CeNT can be used to deliver small non-permeable molecules to CD34+ cells and encouraged us to attempt delivering larger molecules which can be used to modify and regulate gene expression.

Efficient RNA delivery to human HSPCs by CeNT. Next, we investigated whether more complex cargoes can be delivered to CD34+ HSPCs using nanostraws by delivering an mRNA encoding for the fluorescent reporter protein GFP. We chose to deliver mRNA because it is generally well-tolerated by human CD34+ cells, while plasmid DNA delivery has cytotoxic effects on this cell type. A further benefit of using RNA over plasmid DNA is that it does not have to translocate into the nucleus, reducing the biological complexity, and allowing faster detection of the expressed protein. Six hours after treatment, we observed efficient GFP expression in more than 75% of treated CD34+ cells (FIG. 1E). The GFP fluorescence intensity in CeNT treated cells appeared to be uniformly distributed within the treated cell population (FIG. 1E).

To confirm that other functional RNA species can also be delivered using CeNT, we targeted small interfering RNAs (siRNAs) into HSPCs. Small interfering RNAs can be used to temporarily regulate gene expression by degrading a targeted mRNA in the cell. We used siRNAs against STAG2, a subunit of the cohesin complex, which has previously been successfully knocked down with lentivirally delivered shRNAs. Two days after the treatment, qPCR analysis was performed to determine the efficiency of the knock-down. Treatment with two different siRNAs significantly reduced the expression of STAG2 relative to the commonly used reference gene HPRT, while mock treatment or CeNT of a non-targeting siRNA did not affect STAG2 levels (FIG. 1F). Taken together, our results show successful delivery and translation of exogenous mRNA, and also targeted downregulation of endogenous genes with siRNA by CeNT.

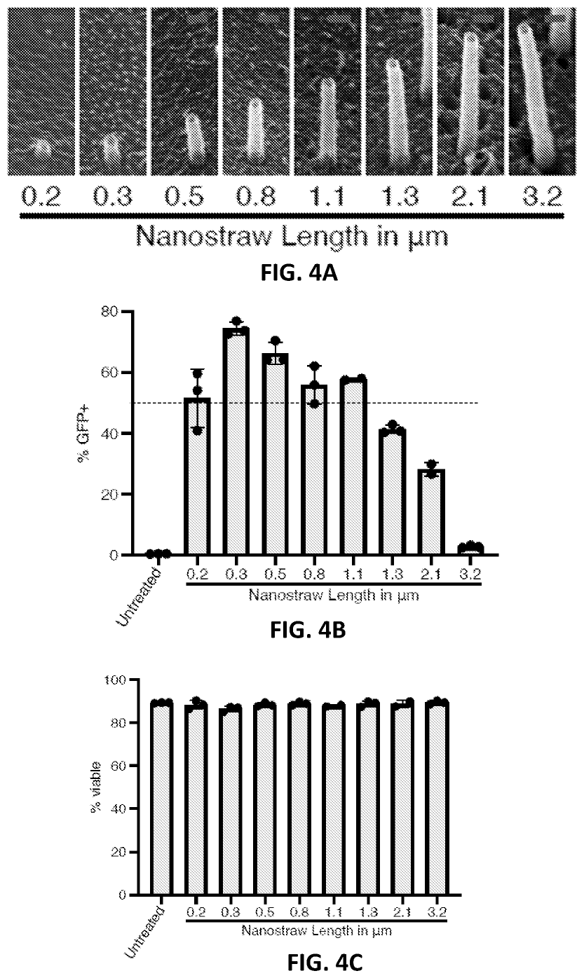

Impact of nanostraw length and cargo size on delivery efficiency. After establishing nanostraw-mediated intracellular access to HSPCs, we investigated whether nanostraw length and cargo sizes have an impact on delivery efficiency and cell viability. To identify the appropriate length range, we manufactured nanostraws of different lengths, ranging from 200 nm to 3200 nm (FIG. 4A), and used them to deliver GFP mRNA to CD34+ cells. One day after the delivery we analyzed GFP fluorescence using flow cytometry and found that lengths up to 1100 nm resulted in satisfactory delivery efficiencies of 50% or higher (FIG. 4B). Longer nanostraws performed progressively worse, with 3200 nm length being completely unsuitable for delivery. Nanostraws are manufactured by removing layers of polymer to expose the alumina nanochannels that are already embedded in the membrane. The length of the nanostraws can be increased by removing more polymer, but the total length of the alumina channels is constant. Therefore, we do not expect increased clogging with longer nanostraws, because the cargo has to travel the same distance through the alumina nanochannels, regardless of how long the nanostraws are. It is possible that the reduced rigidity and stiffness of longer nanostraws might prevent successful cell piercing and cause the observed lower delivery efficiencies. Treated cells maintained their viability, as determined by 7AAD and Annexin V staining, compared to untreated cells, regardless of nanostraw length (FIG. 4C), particularly when a higher voltage (e.g., >30 V) was used as described herein. Furthermore, the viability of the cells within this range of lengths, using >30 V, was surprisingly high (e.g., >80%), particularly as compared to previous work in which an increase in voltage applied (e.g., >20 V) using longer (e.g., greater than 1100 nm) nanotubes showed a significant decrease in viability. Thus, FIG. 4B highlights the significant and unexpected improvement in efficiency and survivability when transfecting cells by centrifugation onto nanostraws having a length of between 200-1100 nm at >30 V.

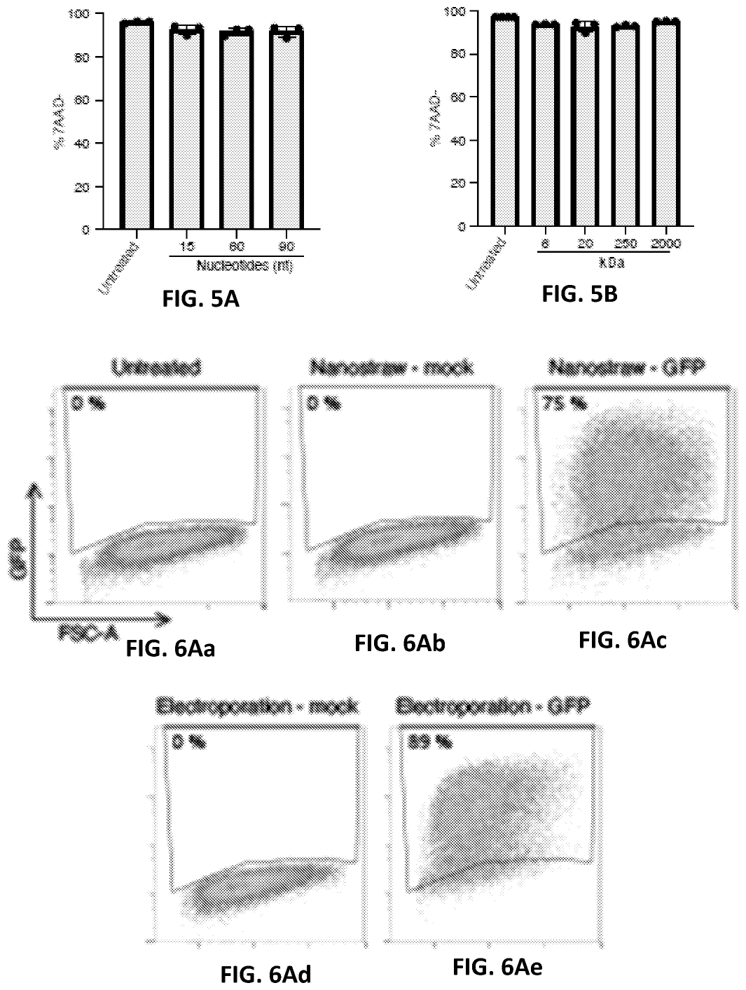

A transfection method is most useful if it can be used to deliver diverse cargos of different sizes. Therefore, we investigated if the efficiency of nanostraw-mediated delivery is affected by the molecular size of the cargo. To this end we used nanostraws to target HSPCs with FITC-tagged DNA oligonucleotides of different lengths and fluorescein-labelled dextran molecules of molecular weights ranging from 6 kDa to 2000 kDa. The delivery efficiency was determined by flow cytometry directly after the cargo delivery and a washing step. DNA oligos could be delivered with average efficiencies ranging from 76% to 80% (FIG. 1G). Delivery of the shortest, 6 kDa dextran was delivered to 83% of treated cells on average. Longer dextrans (20, 250 and 2000 kDa) were delivered with average efficiencies ranging from 61% to 67% (FIG. 1H and FIG. 1I). We also stained treated cells with the dead-cell stain 7AAD and found that there is no immediate toxicity resulting from the delivery of differentially sized molecules (FIG. 5A and FIG. 5B). Taken together, these results indicate that delivery of smaller molecules is slightly more efficient than delivery of larger molecules. Despite this, even extremely large molecules of up to 2000 kDa can successfully be delivered using nanostraws, which confirms that molecular weight is not a limiting factor for CeNT within the tested size range.

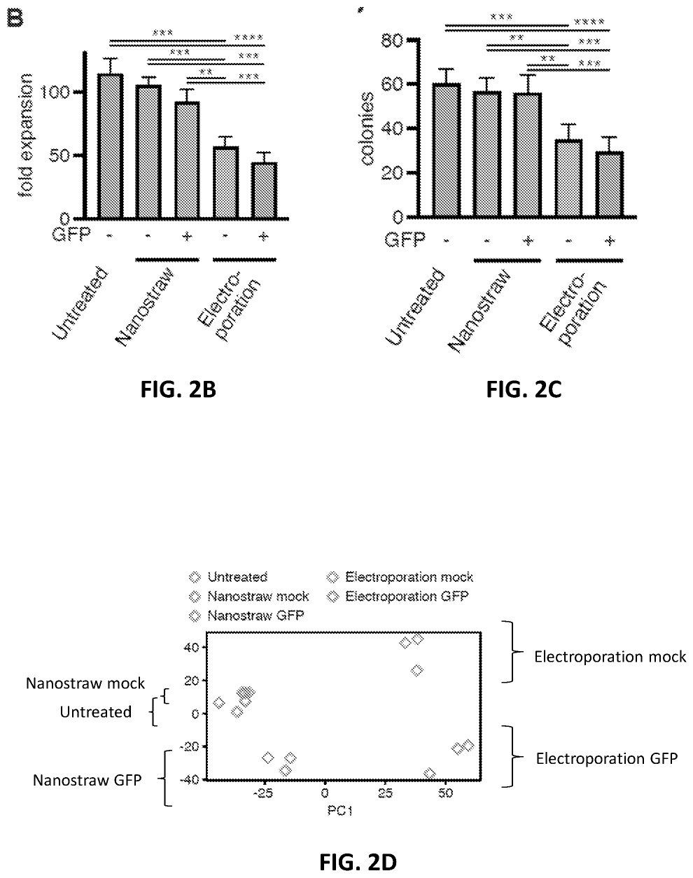

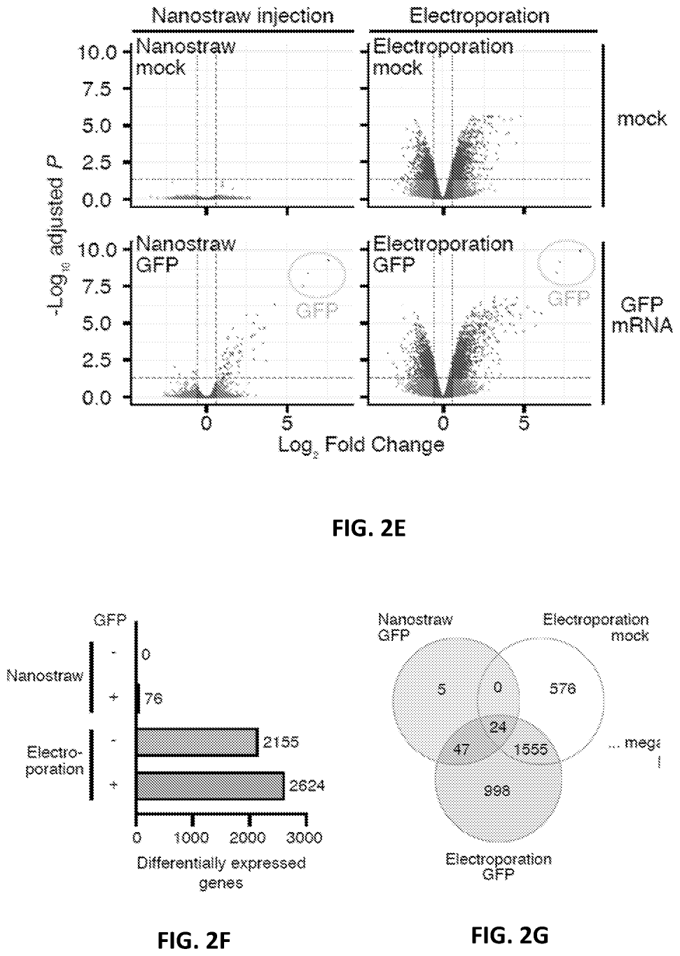

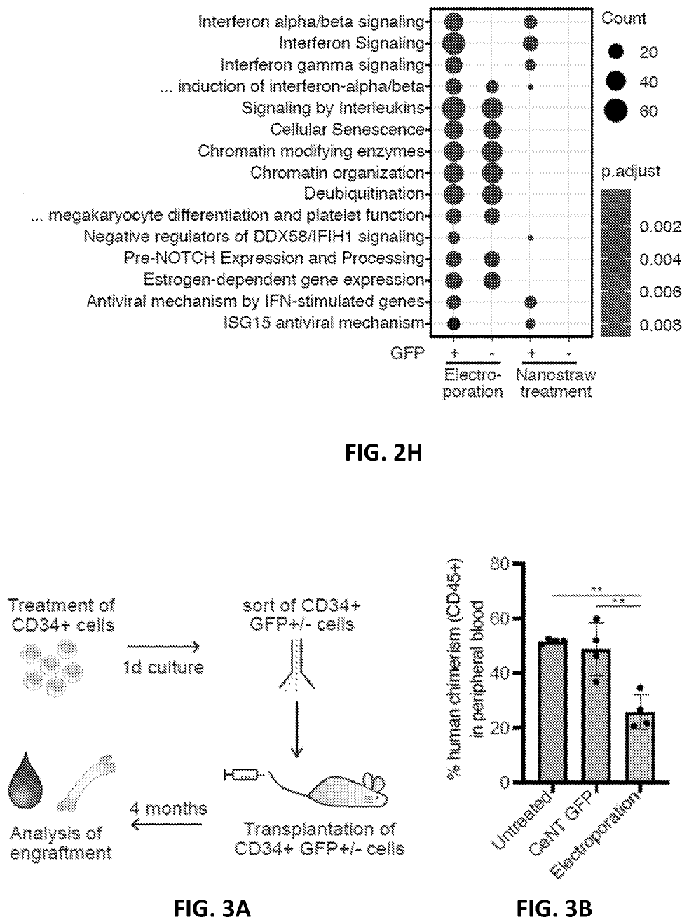

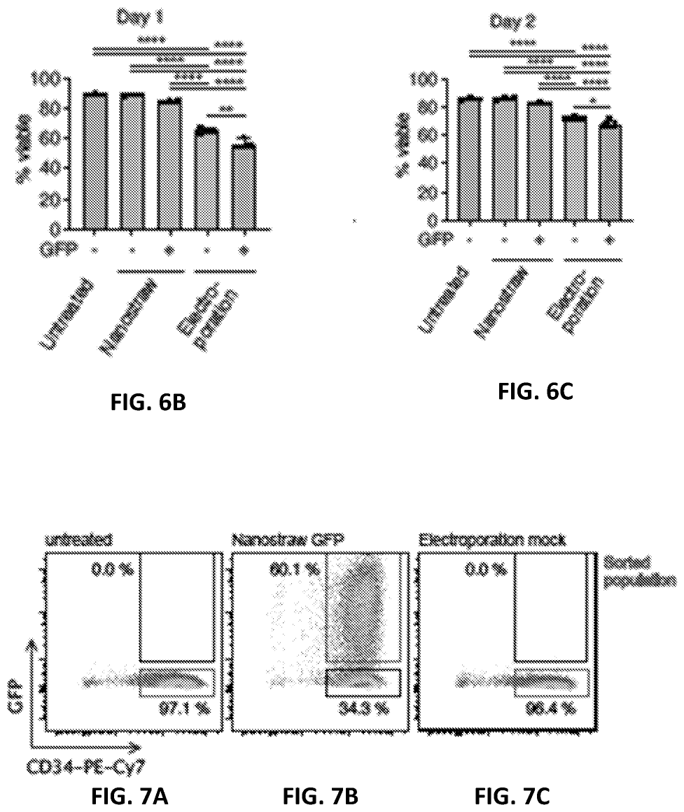

HSPCs are fully viable and functional after CeNT treatment. In the next experiment, we performed a more detailed investigation into the effect of CeNT on cell viability and function, and compared it directly with conventional electroporation using a commercially available kit specifically designed for CD34+ cells (FIG. 2A). We subjected the same batch of HSPCs to CeNT based delivery of GFP mRNA or mock treatment, as well as conventional electroporation with GFP mRNA or mock (FIG. 6Aa-FIG. 6Ae). The same electric pulse was applied in all mock conditions, but without mRNA present in the delivery buffers. All conditions were performed in parallel and in triplicates, and the cells were cultured under the same conditions (cell concentration, medium), in parallel to untreated cells. After 6 hours, live CD34+GFP+ or CD34+GFP− cells were sorted for continued culture, colony forming cell (CFC) assays, and gene expression analysis. We also cultured unsorted cells of each condition and measured cell viability with 7AAD and Annexin V staining after 1 and 2 days in culture. We found that nanostraw treated cells were equally viable as untreated cells at both timepoints, whereas electroporation led to a strong reduction of viability (FIG. 6B and FIG. 6C).

To assess the proliferative capacity of treated cells, we plated equal numbers of sorted, live cells, and determined the total number of live cells after 2 weeks in culture (FIG. 2 B). Completely untreated cells expanded 115±21-fold during a two-week period, and Nanostraw mock- and Nanostraw GFP-treated cells expanded 106±12 and 93±14-fold, respectively. Electroporated cells (mock and GFP) expanded substantially less, 57±15 and 45±11-fold, respectively. Overall, this shows that the proliferative capacity of CeNT treated cells, in contrast to electroporated cells, is not significantly affected by nanostraw treatment.

To investigate whether CeNT treatment affects the functional capacity of HSPCs to form colonies, we performed a Colony Forming Cell (CFC) assay. Live CD34+ cells were plated on semi-solid, methylcellulose MethoCult medium with differentiation promoting cytokines. After 14 days, the number and type of colonies were determined. Untreated, Nanostraw mock- and Nanostraw GFP-treated cells gave rise to similar numbers of colonies, 60±8, 57±6 and 56±8 respectively. Electroporated cells, both with and without GFP, showed greatly reduced colony forming capability (35±8, 30±7), even though the same number of live cells was plated (FIG. 2C). The colony forming capacity was thus significantly lower in both electroporation settings, compared to untreated and nanostraw treated cells. Additionally, nanostraw treated cells and untreated cells gave rise to similar sized colonies, whereas electroporated cells resulted in smaller and less dense colonies. This is in line with previously published data that show more than 75% reduction of colony forming capacity in human CD34+ cells upon electroporation. In summary, the ability of CD34+ progenitor cells to mature and form colonies is not affected by nanostraw treatment, which is key for clinical applications and functional studies.

CeNT treatment does not perturb gene expression. To detect more subtle effects of CeNT on the cells, we performed global gene expression analysis. Six hours after treatment, we sorted CD34+GFP+ or CD34+GFP− cells and performed microarray analysis. We used Principal Component Analysis (PCA) to visualize how the gene expression compared between the different treatments. Both mock and GFP CeNT treated cells, unlike electroporated cells, clustered together closely with untreated cells in the PCA (FIG. 2D). This indicates that the gene expression profiles of nanostraw treated HSPCs are very similar to untreated HSPCs.

Remarkably, we did not detect any significantly up- or downregulated genes in response to mock nanostraw treatment (FIG. 2E and FIG. 2F). This shows that subjecting CD34+ cells to nanostraw treatment does not cause a strong change at the transcriptional level, further corroborating the previous results, which show that cell viability and function are not perturbed. When delivering GFP mRNA using nanostraws, 76 genes were up- or downregulated. Electroporation had a strong impact on global gene expression, with over 2000 genes affected, regardless of whether GFP mRNA was present or not (FIG. 2F). Of note, 71 out of 76 differentially regulated genes in the Nanostraw GFP delivery were also differentially regulated in the Electroporation GFP condition (FIG. 2G), indicating that these genes are specifically associated with the presence of GFP mRNA.

Pathway enrichment analysis showed that differentially regulated genes in the Nanostraw GFP condition were mostly related to response mechanisms to foreign RNAs, such as interferon signaling (FIG. 2H). These pathways were also enriched in the Electroporation GFP condition, but not the Electroporation Mock condition, supporting the notion that these pathways are related to the delivery of exogenous GFP mRNA. In both electroporation conditions, several pathways related to cellular stress were upregulated. Importantly, we did not see any upregulation of these stress related pathways in nanostraw treated cells. This may be one of the reasons for the better cell function of nanostraw treated cells over electroporated cells. Moreover, the absence of differentially expressed genes from CeNT is of great benefit for studying the immediate effect of introduced biomolecules, without any interfering signals from the delivery method.

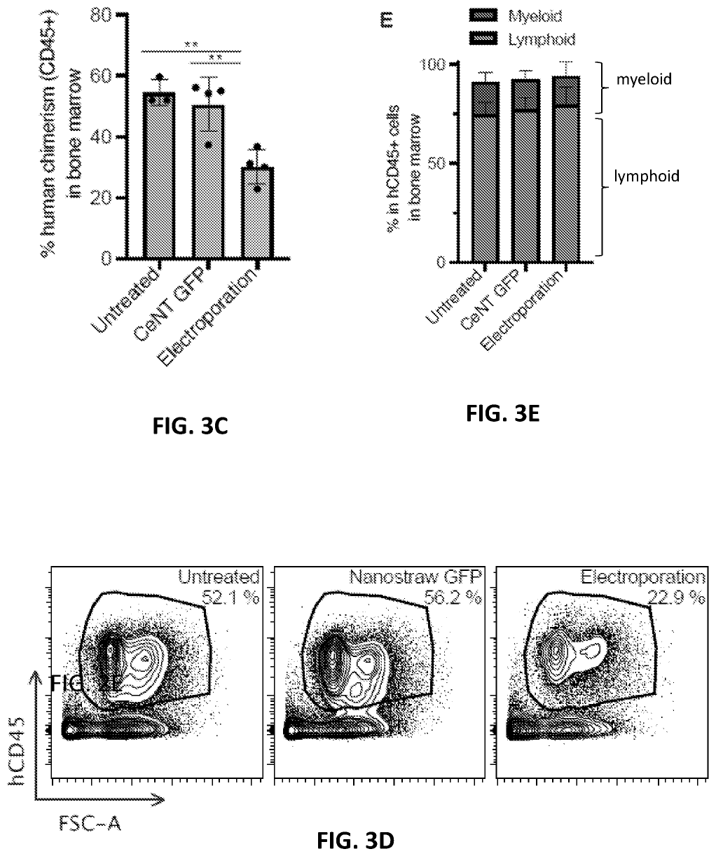

CeNT treated HSPCs engraft in immunocompromised mice. Our previous in vitro experiments confirmed that hematopoietic progenitor cells are not negatively affected by CeNT. Next, we wanted to investigate the impact of CeNT on functional, long-term repopulating HSCs and their multi-lineage engraftment potential in vivo. CD34+ cells were treated with nanostraw injection and mock electroporation. After 1 day in culture, live CD34+GFP+ or CD34+GFP− cells were sorted and injected in equal numbers into sub-lethally irradiated, immunocompromised mice (FIG. 3A). For the nanostraw treated cells, only GFP+ cells were sorted to ensure that only cells that were successfully targeted with the nanostraws were transplanted (FIG. 7A-FIG. 7C). After 4 months, the percentage of human cells in the peripheral blood was 52±1% in the untreated and 49±10% in the nanostraw treated condition, indicating that the engraftment potential of human HSPCs is not impaired by nanostraw treatment. Cells treated with conventional electroporation had significantly lower engraftment potential with 26±6% (FIG. 3B). Similar results were observed in the bone marrow (FIG. 3C and FIG. 3D). Nanostraw treated cells gave rise to both myeloid and lymphoid cells, and we observed a similar lineage distribution in all conditions (FIG. 3E). This further confirms that the functional properties of human HSPCs are maintained upon CeNT.

Conclusion. Human HSPCs are recalcitrant to transfection. Currently existing methods for nucleic acid delivery, such as electroporation, have greatly expanded our knowledge of HSC biology but have limitations with regard to safety, toxicity, or preservation of regular cell function. We show that CeNT is an efficient alternative method in the transfection toolbox that can be used for delivering non-permeable small molecules, siRNA, and mRNA to primary human HSPCs. CeNT delivered RNA species execute their expected biological function, and the treatment is exceptionally gentle and does not cause any functional impairment. Human HSPCs that were treated with CeNT completely retained their proliferative capacity and colony forming potential. They also engraft in immunocompromised mice with similar efficiency as untreated cells and result in long-term and multi-lineage blood production. This confirms that even naïve and undifferentiated HSCs, which are particularly difficult to target, can be targeted using CeNT.

The gentle nature of this delivery method is further underlined by the minimal impact on the gene expression of treated cells. While conventional bulk electroporation causes dysregulation of several thousand genes, global gene expression is virtually unaffected by CeNT. This allows for the detection of subtle changes in gene expression that are caused purely by the presence of the introduced RNA. Such changes are at risk of being masked by the profound effects on gene expression that other methods have. CeNT makes it possible to study the immediate effect of transient mRNA delivery on primary cells, without having interfering signals from the transfection method. This is useful for elucidating subtle mechanisms and pathways that might remain hidden otherwise.

Limited cell numbers make it difficult to study the biology of primary cells. Any treatment of such cells should not reduce the already low number of available cells, and also not impair their function. This becomes especially important when working with patient-derived cells, which are even more sensitive to stress. CeNT is an efficient method for targeting sensitive primary stem cells, and we expect it to be useful for both clinical applications and functional studies, where maximal cell recovery, viability, and performance are essential.

Production and assembly of nanostraws. Nanostraws were developed in close collaboration with Navan Technologies Inc. Track-etched polyethylene terephthalate (PET) membranes (GVS, Sanford, USA) with pore diameter of 100 nm, pore density of 3*10⁷ cm−2, and membrane thickness of 12 μm were used as templates for nanostraw fabrication. The membranes were coated in a Savannah S100 Atomic Layer Deposition reactor (Cambridge Nanotech, Cambridge, MA, USA) using trimethylaluminum and water as precursors. About 10 nm aluminum oxide was deposited. The top layer aluminum oxide was removed using Inductively Coupled Plasma Reactive Ion Etching (ICP-RIE) in an APEX SLR plasma etcher (Plasma Therm, St. Petersburg FL, USA) using an Ar plasma at 100 W RF, 250 W ICP for 2 min. Oxygen based RIE was used to form the nanostraws by removing some of the PET membrane using 25 W RF, 500 W ICP for 90 s. Successful processing was confirmed by cutting out a small piece of the nanostraws membrane which was coated by 4 nm Pd:Pt and imaged by Scanning Electron Microscopy (SEM) in a LEO 1560 SEM (Zeiss, Oberkochen, Germany) using a thermal field emission gun operating at 15 kV. The processed nanostraw membranes were attached to the bottom of cell culture compatible plastic tubes using double sided tape in order to form a cell culture chamber. The tubes had an inner diameter of 5 mm and could hold up to 300 μL of liquid. The assembled cell culture chambers were sterilized in a custom-built UV/Ozone box for 5 min. For the nanostraw length series, the following processing steps were performed: After ALD coating as described above, the membrane (same batch) was cut into smaller pieces for oxygen RIE. Oxygen RIE etching times ranging from 1s to 360s were used to form nanostraws of lengths from 200 nm to 3200 nm. The nanostraw length was estimated from 30° tilted view SEM images of nanostraws standing up on their substrate (for lengths up to 1 μm). Due to charging effects, longer nanostraws longer than 1 μm had to be broken and imaged while lying down on the substrate.

Cell culture. Umbilical cord blood was collected at Skåne University Hospital and Helsingborg Hospital. Mononuclear cells were extracted using Lymphoprep tubes (Alere Technologies AS, #1019818). CD34+ cells from mixed donors were isolated from mononuclear cells with CD34 MicroBead Kit (Miltenyi Biotec, #130-046-703) according to the manufacturer's instructions and cryopreserved in FBS supplemented with 10% DMSO. CD34+ cells were cultured in StemSpan SFEM (Stemcell Technologies, #09650) with human SCF (PeproTech, #300-07), human TPO (PeproTech, #300-18), and human Flt3L (PeproTech, #300-19), at 100 ng/mL each, at 37° C. and 5% CO2.

CeNT treatment. For experiments in FIG. 2A-FIG. 2H, CD34+ cells were thawed and cultured for 1 d. The cells were split and treated with the following conditions: Nanostraw mock, Nanostraw GFP, Electroporation mock, Electroporation GFP, and completely untreated. Each condition was performed in separate, independent triplicates. For each nanostraw treatment, 150 000 cells were centrifuged (600 g, 3 min, 20° C.) onto the nanostraw membrane. A droplet containing the cargo was placed on top of an Au coated glass slide. For the mock condition, the membrane was placed on top of a 10 μL 0.1× PBS droplet, and for the GFP condition onto a 10 μL 0.1× PBS droplet containing 2 μg EGFP mRNA (L-7601, TriLink BioTechnologies). A pulsed square electric field (40 V, 40 Hz, 200 μs, 3×40s) was applied in between the Au slide and a Pt electrode dipped into the cell culture container using a Grass S48 Stimulator (Astro-Med GmbH, Rodgau, Germany). The applied voltage at the Pt electrode and the current flowing through were monitored using an oscilloscope (PICOSCOPE 2206B, Pico Technology, UK). The Pt electrode was kept at positive bias in order to help drive negatively charged mRNA through the nanostraws and into the cells. For each electroporation treatment, 1*10⁶ cells were either electroporated with GFP mRNA, or mock electroporated without any cargo, as described in more detail below. Cells were then cultured with exactly the same cell concentration in each condition. After 6 h, live 7AAD-CD34+GFP+, or GFP− for mock conditions, cells were sorted for a proliferation assay, a CFC assay, and microarray analysis. Each individual treatment condition was sorted, before the next replicates were sorted, to account for any time-dependent effects. During the sort, the efficiency of the GFP delivery was recorded.

Scanning electron microscopy (SEM). For SEM of HSPCs on nanostraws, additional sample preparation was needed. HSPCs were centrifuged onto the nanostraw membrane followed by three times rinsing in PBS to remove cell media. The cells were then fixed in 2.5% glutaraldehyde. The next day, the cells were dehydrated in an EtOH drying series of 10 min each in 30%, 50%, 75%, 90%, 95%, 99.5% EtOH. Critical point drying (CPD) was used to completely remove the EtOH from the cells with minimal disruption of the cellular shape in a Quorum K850 CPD (Quorum technologies, Lewes UK). The CP dried samples were coated with 4 nm Pd:Pt and imaged with a LEO 1560 SEM (Zeiss, Oberkochen, Germany).

Propidium Iodide delivery. Propidium Iodide (PI) was diluted to 200 μg/mL in 0.1× PBS and 10 μL were placed on the bottom electrode. CD34+ cells were centrifuged (600 g, 3 min) onto the nanostraw membrane and placed onto the PI containing droplet. A pulsed electric field (20-30 V, 40 Hz, 200 μs, 2× 40s) was applied to drive the PI delivery into the cytoplasm of the cells. After the treatment, the bottom of the nanostraw membrane was rinsed, and the cells were directly imaged with an Olympus IX70 fluorescence microscope. The delivery efficiency was estimated by dividing the number of PI-positive cells (red) by the total number of cells that were counted in an image taken at 40× magnification. As a negative control, the experiment was performed with a 10 μL 0.1×PBS droplet, without any PI, placed below on the bottom electrode, and the same absolute amount of PI that was used in the previous delivery added directly into the cell containing medium. After application of the pulsed electric field, cells were imaged as above.

Dextran and DNA oligo delivery. FITC labelled DNA oligos (15 nt, 60 nt, and 90 nt) with the following random sequences were ordered from Integrated DNA Technologies, Inc. 15 nt: ACTGGTCAAC/iFluorT/GGTC; 60 nt: ACTGGTCAAC/iFluorT/GGTCATCCTGAAGT AAATGCTATGCGACTGATTGGGCTACGCTCCGCTA; 90 nt: ACGCGGCTAGGG ACTGGTCAAC/iFluorT/GGTCATCCTGAAGTAAATGCTATGCGACTGATTGGGCTACG CTCCGCTAAAAAAGTGCTAAAGGCAG. They were delivered by placing a 10 μL droplet 0.1× PBS containing 1 nmol DNA oligo under the nanostraw membrane and applying a pulsed electric field (40V, 40 Hz, 200 μs, 3×40s). Fluorescein-dextran (6 kDa, 20 kDa, 250 kDa, and 2000 kDa) was purchased from Fina Biosolutions, LLC, and delivered by placing a 10 μL H2O droplet containing 1 μg/μL fluorescein-dextran under the nanostraw membrane and applying a pulsed electric field (40V, 40 Hz, 200 μs, 3×40s).

Electroporation. Electroporation was performed using the Nucleofector 2b Device (Lonza) and the Human CD34+ Cell Nucleofector Kit (VAPA-1003, Lonza) according to the manufacturer's instructions. For each electroporation, 1*10⁶ cells were treated. In conditions where GFP was delivered, 10 μg Cleancap EGFP mRNA (L-7601, TriLink BioTechnologies) was added to the electroporation mix.

siRNA delivery. We designed a siRNA against STAG2 with the target sequence GCAGUUCUUACAGCUUUGUUU (siRNA1). It was synthesized together with two predesigned siRNAs (siRNA2-STAG2: SASI_Hs02_00311139, siRNA3 non-targeting control: SIC001) by Sigma Aldrich/Merck. 30 000 CD34+ cells were centrifuged (600 g, 3 min) onto a nanostraw membrane and placed onto a 10 μL 0.1× PBS droplet containing 20 pmol siRNA and exposed to a pulsed electric field (40V, 40 Hz, 200 μs, 3×40s). Each siRNA delivery was performed in triplicates. Two days after the treatment, RNA was extracted from the cells using the RNeasy Micro Kit (Qiagen) and reverse-transcribed using the SuperScript IV First-Strand Synthesis System (Thermo Fisher Scientific). qPCR was performed with TaqMan assays for STAG2 (Hs00198227_m1) and HPRT (Hs99999909_m1) using the 7900HT FastReal-Time PCR System (Applied Biosystems).

Flow cytometry/FACS. The following anti-human antibodies were used for staining. Sort for microarray, long-term culture, and CFC assay: CD34-A700 (BD Pharmigen #561440, Clone 581). Bone marrow and peripheral blood engraftment analysis: CD45-APC (Biolegend #304037, Clone HI30), CD33-PE (Biolegend #303404, Clone WM53), CD19-BV605 (BD Horizon #562653 Clone SJ25-C1), CD3-PE-Cy7 (Invitrogen #25-0038-42, Clone UCHT1). 7AAD (Sigma-Aldrich, #A9400) was used to discriminate dead cells. Stained cells were analyzed with BD LSRFortessa or BD FACSCanto, or sorted with BD FACSAria Ilu or BD FACSAria III. Data was analyzed with FlowJo 10.6.1 (BD).

Cell viability assay. Cell viability was determined using the 7AAD/PE Annexin V Apoptosis Detection KIT I (BD Biosciences, #559763) according to the manufacturer's instructions.

Proliferation assay. For each sample, 300 live human CD34+GFP+/− cells (n=3) were seeded in a 96 well U-bottom plate. After 2 weeks in culture, total live cell numbers were determined with flow cytometry.

Colony forming assay. 167 live human CD34+GFP+/− cells were sorted into 1 mL MethoCult H4230 (Stemcell Technologies, #04230) with 20% IMDM, 25 ng/mL hSCF, 50 ng/mL GM-CSF, 25 ng/mL IL3 and, 2 U/ml EPO. The cells were cultured in 6-well plates for 2 weeks at 37° C. and 5% CO2. Colonies were counted and scored by an experienced person who was blinded to the sample identity.

Microarray. 20 000 live, human CD34+GFP+/− were sorted into lysis buffer and immediately snap-frozen at −80° C. Total RNA was extracted with the RNeasy Micro Kit (QIAGEN) according to the manufacturer's protocol. Purity and integrity of the RNA was assessed with the Agilent 2100 Bioanalyzer with the RNA 6000 Pico LabChip reagent set (Agilent). Sample preparation and processing with Affymetrix Human Gene 2.0 arrays were performed at an Affymetrix Service Provider and Core Facility, “KFB—Center of Excellence for Fluorescent Bioanalytics” (Regensburg, Germany).

Microarray data analysis. Microarray data was normalized with the Robust Multiarray Average (rma) function of the R package oligo version 1.49.0 and annotated with ENTREZIDs from the Bioconductor annotation data package org.Hs.eg.db version 3.8.2, using the AnnotationDbi package version 1.47.0. Principal component analysis was performed with the prcomp function of the stats package version 3.6.0 and plotted with the ggplot2 package version 3.1.1. Differentially expressed genes (FC>1.5, adjusted p-value<0.05) were detected using the limma package version 3.41.2, and volcano plots were created using the Enhanced Volcano package version 1.3.0. Pathway enrichment analysis of upregulated genes was performed using clusterProfiler version 3.13.0.

Animal experiments. Cord blood-derived human CD34+ cells were thawed and cultured for 1 d, before they were divided for the following treatment conditions: untreated, electroporated with Human CD34+ Cell Nucleofector Kit (VAPA-1003, Lonza), and CeNT with GFP mRNA. The treated cells were cultured for 1 d. Live (7AAD−)CD34+ and, if GFP was part of the treatment, GFP+ cells were sorted for the following transplantation. For each treatment condition, sub-lethally irradiated (300 cGy) NOD.Cg-Prkdcscid Il2rgtm1Wjl/SzJ. (NSG) mice were injected with 59 000 cells in 500 μL PBS/2% FBS into the tail vein. Bone marrow and peripheral blood were analyzed for engraftment and lineage distribution of human cells with flow cytometry after 4 months.

Statistics. When comparing multiple groups, GraphPad Prism 8 was used to perform One-Way analysis of variance (ANOVA) with Tukey's multiple comparison test. Data in figures is shown as Mean±SD and significance is indicated with asterisks (* p<0.05, ** p<0.005, *** p<0.0005, **** p<0.00005).

Data Availability. Microarray data were deposited in the Gene Expression Omnibus (GEO) database of the National Center for Biotechnology Information (NCBI) under the GEO accession number GSE151027.

Media Change

As mentioned above, any of the methods and apparatuses (e.g., systems) described herein may also include a step of changing the media within x minutes (where x is 60, 45, 40, 30, 25, 20, 15, etc.) of performing the after delivery (via nanostraw as described herein) of the cargo. Preferably the media may be changed within 20 minutes or less (e.g., 15 min or less). Changing the media immediately afterwards, surprisingly even where fresh media was used just before (e.g., within 5 hours, within 4 hours, within 3 hours, within 2 hours, within 1 hour, etc.) the delivery of a cargo by nanostraw, may result in a significant increase in viability.

After delivering cargo to stem cells as described herein, there may be a reduction in cell viability as compared to either untreated or other cell types. Replacement of the cell medium with fresh medium appears to avoid this previously undescribed effect, even where the cells were provided with fresh media just before the procedure. Without being bound by a particular theory, this may be due to changes induced in the medium when applying the electric field using the higher voltages described herein (e.g. 30V+). The higher electric field may induce some water splitting reaction in the cell medium and may consume some cytokines, resulting in lowered cell viability.

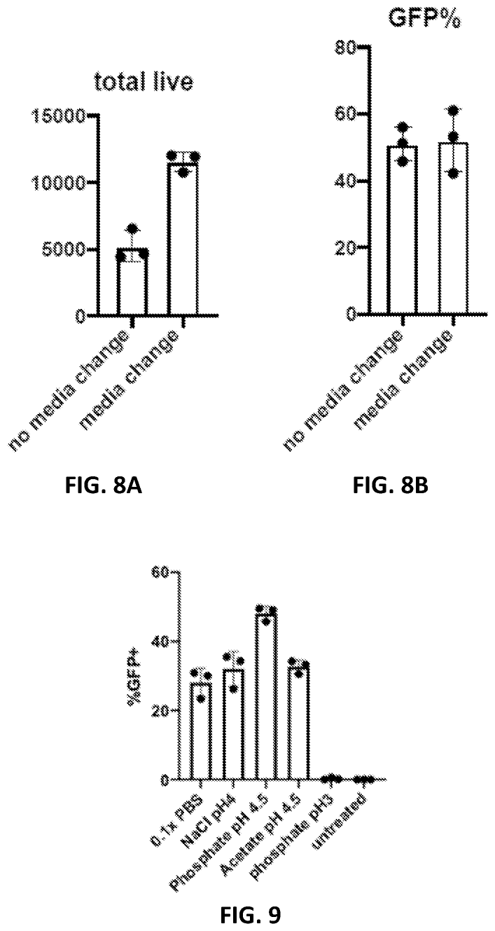

For example, FIGS. 8A and 8B illustrate one example of a comparison of cell viability (FIG. 8A) and expression of transferred material (GFP, FIG. 8B) in cells provided with fresh media immediately (within 20 minutes) after exposure to the electrokinetic voltage to deliver cargo using nanostraws, as described above. FIG. 8A shows the delivery of GFP-mRNA into CD34+ cells using nanostraws; within 15 min after delivery, the cell media was either replaced with fresh media (“media change”) or left with the media that went through the nanostraw delivery process (“no media change”). One day after delivery, total live cell count as well as amount of GFP+ cells was evaluated showing that the cells that went through the media change had a much higher cell viability. As seen in FIG. 8A, there was a significant increase in cell viability for those cells for which the media was changed shortly after the procedure. There was no significant difference in expression of the GFP, as shown in FIG. 8B. In some cases, changing the cell media after this window of time (e.g., after 60 minutes, after 2 hours, after 3 hours, after 4 hours, after 5 hours, after 6 hours, after 8 hours, after 12 hours, after 20 hours, after 24 hours, etc.) or longer did not result in the increase in viability seen when media was changed, e.g., within a short period of time after the application of the voltage.

In general, the methods and apparatuses (e.g., systems including nanostraws) may be used to enhance engraftment of stem cells, as described above, and may be part of a therapy including collecting, treating (using the nanostraws as described herein) and transferring modified cells to a patient. The stem cells may include but are not limited to hematopoietic stem cells. The cells may be modified by the addition of a cargo to the cell as described herein. Cargo may be any one or more compounds/compositions that may enhance engraftment of hematopoietic stem cell, such as cell penetrating peptides, proteins, mRNA, siRNA, miRNA, DNA, sugars, and cardiac specific transcription factors. The cargo may alternatively or additionally include one or more therapeutic agents (proteins, peptides, etc.).

In general, Hematopoietic stem cell gene therapy (HSC-GT) represents an autologous therapeutic intervention by which a normal copy of a deficient gene (a “therapeutic agent”) may be introduced into patient's own HSCs to reestablish effective gene function. As such, HSC-GT bypasses the immunological risks of allogeneic HSC transplantation and the immune suppression needed to prevent or control these risks. HSC-GT offers a curative potential to diseases where hematopoietic cell transplantation is suboptimal (i.e., metachromatic leukodystrophy) or the need for a well-matched donor precludes a significant number of patients from undergoing this therapeutic procedure (i.e., hemoglobinopathies).

As mentioned, in addition to delivering the therapeutic agent in the modified cell(s), the cells may be further modified by the use of nanostraws to enhance engraftment. There are a number of ways to enhance HSC engraftment. For example, neutralizing negative epigenetic regulation by histone deacetylase 5 (HDAC5) increases surface CXCR4 expression and promotes human HSC homing and engraftment (shown, e.g., in immune-deficient NSG mice). Other cargos may include glucocorticoids, pharmacological stabilization of hypoxia-inducible factor (HIF)-1α, increasing membrane lipid raft aggregation, and inhibition of dipeptidyl peptidase 4 (DPP4) to facilitate homing and engraftment of stem cells. Cells may include cargo to modulating the mitochondria permeability transition pore (MPTP) to mitigate ambient air-induced extra physiological oxygen stress/shock (EPHOSS) by hypoxic harvest and processing, or using cyclosporine A during air collection may increase functional HSC numbers and improves HSC engraftment. Thus, any of the methods an apparatuses described herein may be used to modify the stem cells (e.g., hematopoietic stem/progenitor cells) using any of the methods and/or apparatuses described herein to enhance engraftment. Peptides known or suspected to improve engraftment may be used. The cells described herein may be modified by the nanostraws to improve or enhance the responsiveness of the cells (e.g., HSPCs) to homing gradients and/or to increase or improve the tethering of transplanted cells (e.g., HSPCs) to the target region, such as basement membrane (BM) endothelium and subsequently their adhesion, e.g., in the BM microenvironment.

For example, hematopoietic stem/progenitor cells may be transfected or treated using nanostraws as described herein to include or express IGF-1, small molecules such as C3a and/or LL-37 (a cathelicidin fragment). Other potential proteins or polynucleotides encoding these proteins (or variants thereof) may include, but are not limited to CXCR4, type 1 receptor for S1P (S1PR₁) and cell-surface mucins (e.g., P-selectin glycoprotein ligand 1, PSGL-1). Other potential cargos may include Prostaglandin E₂ (PGE₂). Inhibitors of Heme oxygenase 1 (HO-1) may also be used (such, as, but not limited to small-molecule inhibitors of this enzyme, genetic inhibitors such as anti-sense mRNA, etc.). Inhibitors of Glycogen synthase kinase 3β(GSK-3β), such as but not limited to 6-bromoindirubin 3′ oxime (BIO) may also or alternatively be used.

For example, cells (e.g., hematopoietic stem/progenitor cells) may be collected from the patient or donor, and/or pooled from multiple sources, and may be treated as described herein in order to enhance engraftment and/or to express one or more therapeutic molecule. These cells may then be screened and some or all of them may be delivered to a patient as part of a therapy.

In general, in any of the methods and apparatuses described herein the pH (and in particular, the pH of the one or more buffers, including the buffer used for delivery of the cargo), may be optimized. The pH may be, e.g., between 4-6 for some cargo. FIG. 9 is a graph illustrating the effect of buffer pH on the methods as described herein. In FIG. 9, eGFP expression after delivery of eGFP-mRNA using cargo buffers of different pH is illustrated. The standard buffer (0.1× PBS) was found to be not as good as phosphate buffer pH 4.5. pH 4.5 is not necessarily an optimal pH, but shows that varying pH in the cargo container can be beneficial and significantly increase delivery/expression efficiency.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

FITC labelled DNA oligos (15 nt) with the
following random sequences were ordered from
Integrated DNA Technologies, Inc.
SEQ. ID No. 1
ACTGGTCAAC/iFluorT/GGTC
FITC labelled DNA oligos (60 nt) with the
following random sequences were ordered from
Integrated DNA Technologies, Inc.
SEQ. ID No. 2
ACTGGTCAAC/iFluorT/GGTCATCCTGAAGTAAATGCTATGCGACTG
ATTGGGCTACGCTCCGCTA
FITC labelled DNA oligos (90 nt) with the
following random sequences were ordered from
Integrated DNA Technologies, Inc.
SEQ. ID No. 3
ACGCGGCTAGGGACTGGTCAAC/iFluorT/GGTCATCCTGAAGTAAAT
GCTATGCGACTGATTGGGCTACGCTCCGCTAAAAAAGTGCTAAAGGCAG
siRNA1
siRNA against STAG2
SEQ. ID No. 4
GCAGUUCUUACAGCUUUGUUU

1. A method of delivering a biologically relevant cargo, the method comprising:
centrifuging primary stem cells to drive the primary stem cells into contact with a plurality of nanostraws, wherein the plurality of nanostraws extend through a substrate and a distance beyond the substrate that is between 200 nm and 1100 nm, further wherein the plurality of nanostraws are hollow and have an inner diameter of between 5 nm-500 nm; and
driving the cargo from the nanostraws into an intracellular volume of the cells with a pulsed electrical field comprising at least 30 volts across the cells, so that at least 80% of the cells are viable and at least 20% of the cells take up the biologically relevant cargo.

2. The method of claim 1, further comprising replacing a cellular media for the cells within 20 minutes of applying the pulsed electrical field.

3. The method of claim 1, further comprising replacing a media for the cells within 20 minutes of applying the pulsed electrical field, even where the media was applied before performing the driving step.

4. The method of claim 1 wherein the primary stem cells are isolated hematopoietic stem cells.

5. The method of claim 1 wherein applying comprises applying a pulse electrical field comprising at least 35 Volts across the cells.

6. (canceled)

7. The method of claim 1 wherein the membrane comprises an areal density of at least 1*10⁷/cm2.

8. The method of claim 1 wherein the membrane comprises an areal density of at between 2*10⁷/cm² and 4*10⁷/cm².

9. The method of claim 1 wherein the nanostraws have a diameter less than 500 nm.

10. The method of claim 1 wherein the nanostraws have a diameter less than 200 nm.

11. The method of claim 1 wherein the nanostraws have a diameter between 50 nm-200 nm.

12. The method of claim 4 wherein the hematopoietic stem cells comprise CD34+ cells.

13. The method of claim 1 wherein the cargo comprises nucleic acid or protein.

14. (canceled)

15. (canceled)

16. The method of claim 1 wherein the cargo comprises mRNA or siRNA.

17. The method of claim 1 further comprising delivering the stem cells to an animal or human patient.

18. (canceled)

19. The method of claim 1 further comprising delivering the stem cells to the bone marrow of an animal or human patient.

20-22. (canceled)

23. The method of claim 1 further wherein less than 5% of genes in control primary stem cells are nonspecifically activated when compared with untreated primary stem cells.

24. The method of claim 1 further wherein the primary stem cells maintain substantially normal gene expression activity as measured.

25. The method of claim 1, wherein the cargo comprises one or more compounds to enhance engraftment.

26. The method of claim 1, wherein the primary stem cells are driven into contact with a plurality of nanostraws within 24 hours of isolation.

27. A method of enhancing engraftment, the method comprising:
centrifuging primary stem cells to drive the primary stem cells into contact with a plurality of nanostraws, wherein the plurality of nanostraws extend through a substrate and a distance beyond the substrate that is between 200 nm and 1100 nm, further wherein the plurality of nanostraws are hollow and have an inner diameter of between 5 nm-500 nm; and
driving a cargo comprising one or more compounds to enhance engraftment from the nanostraws into an intracellular volume of the cells with a pulsed electrical field comprising at least 30 volts across the cells, so that at least 80% of the cells are viable and at least 20% of the cells take up the biologically relevant cargo.
